(19) United States
(12) Patent Application Publication
(10) Pub. No.: US 2006/0234304 A1
(43) Pub. Date: Oct. 19, 2006

(54) Diagnosis of acute myocardial, ischemic diseases

(75) Inventors: Ildiko Amann-Zalan, Weinheim (DE); Jurgen Spinke, Lorsch (DE)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Roche Diagnostics Operations, Inc

(21) Appl. No.: 11/417,978

(22) Filed: May 3, 2006

Related U.S. Application Data
(63) Continuation of application No. PCT/EP04/12259, filed on Oct. 29, 2004.

(30) Foreign Application Priority Data
Nov. 3, 2003 (DE) 10351238.1

(51) Int. Cl. G01N 33/53

(52) U.S. Cl. 435/7.1

(57) ABSTRACT

A method for the diagnosis of acute myocardial, ischaemic diseases, for example acute myocardial infarct, particularly without increasing ST-paths in EKG (NSTEMI). At least two markers on a patient, who is to examined, are determined. A kit for carrying out said diagnostic method, including a test strip for carrying out quick tests.

### CROSS-REFERENCE TO RELATED APPLICATION

This application is a continuation of PCT Application No. PCT/EP2004/012259, which has an international filing date of Oct. 29, 2004.

### BACKGROUND OF THE INVENTION

In one aspect, the present invention concerns a method for the diagnosis of acute myocardial, ischemic diseases such as acute myocardial infarction, especially without an ST segment elevation in the ECG (NSTEMI) in which at least two markers are determined on a patient to be examined. In addition a kit is provided to carry out the diagnostic method and in particular a test strip for carrying out rapid tests.

The diagnosis of acute myocardial infarction is currently made on the basis of three criteria: The presence of changes in the ECG, chest pain and abnormally elevated cardiac enzymes. It is nevertheless difficult to make a reliable diagnosis since 60% of patients with acute myocardial infarction have no changes in the ECG and 33% of patients do not have typical chest pain. As a result the determination of cardio-specific markers such as troponin T and B-type natriuretic peptide (BNP) is being increasingly used to diagnose acute myocardial infarction (especially in the case of NSTEMI). An increase in the concentration of one of these markers is associated with an increase in the probability of ischemic events including death. This is described for example in the publications of Hamm et al. (New Engl. J. Med. 327 (1992), 146-150), Hamm et al. (New Engl. J. Med. 340 (1999), 1623-1629), Heeschen et al. (The Lancet 354 (1999), 1757-1962), Klootwijk and Hamm (The Lancet 353, Suppl. II (1999), 10-15), Wei et al. (Circulation 88 (1993), 1004-1009), De Lemos (New Engl. J. Med. 345 (2001), 1014-1021).

In De Winter et al. (Cardiovasc. Res. 42 (1999), 240-245) and De Winter et al. (Clin. Chem. 46 (2000), 1597-1603) it has already been found that CRP and troponin I or troponin T are two independent markers for the risk stratification of patients with acute coronary syndrome.

A method is described in EP 03 010 818.7 for diagnosing myocardial infarction or/and for the risk stratification of the acute coronary syndrome in which at least three different cardiospecific markers are determined, where in each case at least one neurohormonal marker, at least one cardiospecific ischemic marker such as troponin T or troponin I and at least one inflammatory marker such as C-reactive protein (CRP) is determined.

The U.S. Pat. No. 6,461,828 discloses the use of a combination of cardiospecific chemical markers such as troponin I and troponin T together with cardiospecific neurohormonal markers such as ANP, ProANP, BNP and ProBNP for the diagnosis and risk stratification of patients who suffer from congestive heart failure (CHF).

WO 02/089657 discloses a method for diagnosing a myocardial ischemia in which BNP or a marker related to BNP such as ProBNP and optionally an additional diagnostic marker such as creatine kinase e.g. CK-MB are determined as markers.

WO 2004/059293 (published on 15 Jul. 2004) concerns diagnostic methods which include a method for the differential diagnosis of heart failure and arterial fibrillation in which BNP or a related peptide such as ProBNP, troponin I or/and troponin T, myoglobin or/and CK-MB are determined as markers.

However, a disadvantage of the known diagnostic methods is that it is not possible to reliably detect risk patients. Thus an increase in cardiospecific ischemic parameters such as troponin T or/and I does not occur until relatively late (generally about 4 hours) after an acute coronary event has occurred. If the patient has ambiguous clinical symptoms or ECG results at this stage, it is not possible to make an unequivocal diagnosis and initiate treatment until a relatively late time. Other ischemic markers such as ischemia-modified albumin (IMA) or muscle-specific markers such as myoglobin are elevated at an earlier time, but only have a relatively low cardiospecificity. Neurohormonal markers such as proBNP are not specific for ischemia according to the current state of knowledge although they have a high cardiospecificity in the case of myocardial infarction.

Hence the inventors have recognized a need in the prior art to develop a method for diagnosing acute myocardial, ischemic diseases such as acute myocardial infarction which enables an earlier and better detection of acute coronary events.

### SUMMARY OF THE INVENTION

It is against the above background that the present invention provides a method for diagnosing acute myocardial, ischemic diseases, where the method includes at least two markers on a patient to be examined, where in each case at least one cardiospecific neurohormonal marker and at least one non-cardiospecific ischemic marker or/and non-cardiospecific muscle-specific marker is determined. In addition it is optionally possible to additionally determine other markers and in particular at least one non-cardiospecific marker for platelet activation or/and a cardiospecific ischemic-necrotic marker.

It was surprisingly found that the combined determination of a cardiospecific neurohormonal marker and at least one of the said non-cardiospecific markers leads to an improved early detection of acute coronary events and thus allows a diagnosis or prognosis even before there is an increase in cardiospecific ischemic parameters. This can prevent unnecessary hospitalization or long periods of stay in the emergency department or in the intensive care unit.

An advantage which results from this is that when acute cardiac events occur such as an acute myocardial infarction and in particular NSTEMI, a larger number of patients can be identified and adequately treated at an earlier time than with the current diagnostic procedure. Hence the combination according to one aspect of the invention of at least two different markers in the diagnosis can reduce the incidence of deaths and other cardiac complications.

“Cardiospecific” in the sense of the one aspect of the invention is understood as a marker whose increase is unequivocally associated with a cardiac disease and is mainly secreted from the heart. In contrast, a “non-cardiospecific” marker is not clearly associated with a cardiac disease and can also be increased in non-cardiac diseases.

The method according to one aspect of the invention comprises the determination of at least two markers, where at least one cardiospecific neurohormonal marker and at least one non-cardiospecific ischemic marker and at least one non-cardiospecific muscle-specific marker are determined.

The neurohormonal marker can for example be selected from atrial (A-type) natriuretic peptide (ANP), B-type natriuretic peptide (BNP) or N-terminal fragments of the respective propeptides NT-proANP and NT-proBNP. BNP or NT-proBNP is typically determined as the neurohormonal marker.

The following can for example be determined as non-cardiospecific ischemic markers: ischemia-modified albumin (IMA), fatty acid binding protein, free fatty acid, pregnancy-associated plasma protein A, glycogen phosphorylase isoenzyme BB or/and sphingosine-1-phosphate. IMA is typically determined as the non-cardiospecific ischemic marker.

Myoglobin or/and creatine kinase MB (CK-MB) can for example be determined as the typical non-cardiospecific muscle-specific markers. Myoglobin or CK-MB are typically determined as non-cardiospecific muscle-specific markers.

Furthermore at least one non-cardiospecific marker for platelet activation can be additionally determined in the method according to one aspect of the invention. CD40 is typically determined as a marker for platelet activation.

In addition at least one cardiospecific ischemic-necrotic marker can be additionally determined in the method according to one aspect of the invention. Troponin T or troponin I can for example be determined as the cardiospecific ischemic-necrotic marker. Troponin T is typically determined as the cardiospecific ischemic-necrotic marker.

The combined determination according to one aspect the invention is typically carried out such that the marker is determined in parallel in one or more samples from a patient to be examined. A positive diagnosis for the presence of an acute disease results from a positive diagnosis for at least one of the tested markers and typically a positive diagnosis for at least two markers. The combination of the stated markers can increase the specificity as well as the sensitivity of the test.

One or more samples derived from the patient such as blood or serum samples are typically examined simultaneously or directly one after the other in one or more tests. The determinations are typically carried out on a single patient sample.

The combined determination of the markers can in principle be carried out by any known methods using common commercial tests. Automated analyzers can for example be used for the determination. Alternatively it is also possible to use rapid tests, for example for use in the emergency department, on the ward or intensive care unit, in the outpatient department or doctor's surgery or as a patient self-test.

The markers are usually determined by an immunoassay using antibodies directed against the markers. The determination is typically carried out on one or more test strips on which the reagents used to determine the individual markers are located in one or more zones in a dry form which dissolves after contact with the sample wherein the individual parameters are detected in a detection zone and typically each parameter is detected in separate areas of the detection zone. Several markers are typically determined on a single test strip.

In addition the markers can of course also be determined by liquid tests.

### DETAILED DESCRIPTION

The detection of BNP or NT-proBNP as a neurohormonal marker is described for example by Richards et al. (Circulation 97 (1998), 1921-1929), Struthers (Eur. Heart J. 20 (1999), 1374-1375), Hunt et al. Clin. Endocrinol. 47 (1997), 287-296), Talwar et al. (Eur. Heart J. 20 (1999), 1736-1744), Darbar et al. (Am. J. Cardiol. 78 (1996), 284-287) as well as in EP-A-0 648 228 and WO 00/45176. A typical test is an electrochemiluminescence immunoassay e.g. the ECLIA® test format of Roche Diagnostics GmbH, Mannheim, Germany.

The detection of IMA as a non-cardiospecific marker is described for example by Wu et al. (Journal: MLO Medical Laboratory Observer 35 (2003), 36-38; 40). The detection of myoglobin is for example described by Pentilla et al. (Clin. Biochem. 33 (2002), 647-653). The detection of CD40 is for example described by Heeschen et al. (New Engl. J. Med. 348 (2003), 1104-1111).

With regard to the determination of troponin T as an example of a cardiospecific ischemic marker reference is made to Katus et al. (Mol. Cell. Cardiol. 21 (1989), 1349-1353), Hamm et al., (N. Engl. Med. 327 (1992), 146-150), Ohmann et al. (N. Engl. J. Med. 335 (1996), 1333-1334), Christenson et al. (Clin. Chem. 44 (1998), 494-501) and numerous other publications as well as to EP-A-0 394 819. Examplary tests for the detection of troponin T are electrochemi-luminescence immunoassays e.g. the test formats ELECSYS® Troponin T and ELECSYS® Troponin T STAT from Roche Diagnostics GmbH, Mannheim, Germany.

A typical combination of markers for diagnosing myocardial infarction is a neurohormonal marker, in particular NT-proBNP, a non-cardio-specific ischemic marker, in particular IMA, and at least one non-cardiospecific myocardium-specific marker in particular myoglobin or/and CK-MB. There is a significant risk for myocardial infarction when at least three of the aforementioned markers are positive. This applies in particular also to patients who have a negative value for a cardiospecific ischemic-necrotic marker such as troponin T.

A further typical combination of markers for diagnosing ischemia/unstable angina pectoris is a cardiospecific neurohormonal marker, in particular NT-proBNP, and a non-cardiospecific ischemic marker, in particular IMA.

Another aspect of the invention is a reagent kit for diagnosing acute myocardial diseases and in particular myocardial infarction wherein the reagent kit contains detection reagents for determining at least two markers, where at least one detection reagent is present for a cardiospecific neurohormonal marker and in each case at least one detection reagent is present for a non-cardiospecific ischemic marker or/and for a non-cardiospecific muscle-specific marker. In addition at least one detection reagent can be optionally present for a non-cardiospecific marker for platelet activation or/and a cardiospecific ischemic-necrotic marker.

The reagent kit is typically designed such that it is suitable for carrying out concurrent determinations of the markers and in particular for carrying out determinations on a single patient sample. For this purpose it is expedient to use detection reagents which enable a determination or markers by a single test format for example an enzyme immunoassay, an electrochemiluminescence test, a turbidimetric test or a rapid test on a test strip.

The reagent kit can be designed such that it is suitable for carrying out the determinations on an automated analyzer or as a rapid test. The detection reagents for several markers are typically present on a single test strip.

In one embodiment of the invention a reagent kit in the form of a test strip is provided for diagnosing acute myocardial diseases which contains reagents for determining BNP or NP-proBNP, for determining IMA, optionally for determining myoglobin or/and CK-MB and optionally for determining troponin T or troponin 1, in particular for determining troponin T, or/and for determining CD40.

The method according to one aspect of the invention and the reagent kit according another aspect of the invention can be used to identify patients with acute coronary syndrome and in particular to improve the early detection of acute coronary events and for example to improve the early detection of acute myocardial infarction.

The present invention is partially elucidated by the following examples, which are provided for exemplification purposes only and are not intended to limit the scope of the invention described in broad terms above. All references cited in this disclosure are incorporated herein by reference.

### EXAMPLES

The investigation described in the following proves the assumption that the determination of several markers—in the sense of one aspect of the invention—improves the diagnosis of acute myocardial, ischemic diseases. Using patients with chest pain as an example, it is possible to demonstrate that an earlier diagnosis of a myocardial infarction (MI) or of an ischemic disease is possible by determining several markers in the sense of one aspect of the invention.

Study design: 40 Serum samples from patients who were admitted to the University Clinic in Lübeck with chest pain were investigated retrospectively. The inclusion criteria for group 1 (=MI group) were a negative troponin T value on admission and a positive value 4-6 hours afterwards. Group 2 (=control group) consisted of patients with negative troponin T values on admission and also 4-6 hours afterwards. The samples were selected at random.

The following markers were determined:
NT-proBNP as a cardiospecific neurohormonal marker
IMA as a non-cardiospecific ischemic marker
Myoglobin as a non-cardiospecific muscle-specific marker
CK-MB as a non-cardiospecific muscle-specific marker

Method: The samples were stored and transported at −20° C. NT-proBNP (Cat. No. 03121640) and myoglobin (Cat. No. 1972146) were determined using the corresponding Roche reagent kit on a Roche ELECSYS automated analyzer according to the manufacturer's instructions. CK-MB and IMA were determined on a Hitachi 912 using the Roche CK-MB Kit (Cat. No. 213284) and the Ischemia Technologies ACB kit (Cat. No. 01VAC260) according to the manufacturer's instructions.

Result: The results of the determinations of all markers are summarized in Tables 1A and 1B, below.

### Example 1

Determination of a cardiospecific neurohormonal marker (NT-proBNP) together with a non-cardiospecific muscle-specific marker (CK-MB) for the early identification of patients with MI.

The following cut-off values were used to identify a positive value:

| | |
|---|---|
| NT-proBNP: | 350 pg/ml |
| CK-MB: | 12 U/l |

If one considers the positive values of the two markers individually and in combination the following distribution is found:

| | | | NT-proBNP |
|---|---|---|---|
| | NT-proBNP | CK-MB | and CK-MB |
| Marker | positive values | positive values | positive values |
| MI group (n = 19) | 65% | 35% | 20% |
| control group (n = 20) | 65% | 15% | 0% |

NT-proBNP or CK-MB alone are not specific for the early diagnosis of a later increase of troponin T and thus of a myocardial infarction. However, if one considers the positive values for both markers, then a myocardial infarction could be diagnosed at an early stage, i.e. already on admission, for 20% of the patients.

### Example 2

Determination of all four parameters for the early identification of patients with MI.

The following cut-offs were used in this case to identify a positive value:

| | |
|---|---|
| NT-proBNP: | 125 pg/ml |
| IMA: | 85 U/ml |
| Myoglobin: | 70 ng/ml |
| CK-MB: | 12 U/ml |

18 of the 20 patients in the MI group already had a positive result on admission for at least one of the four parameters. Zero to two markers were increased in 65% of the samples. 3 or 4 markers were above the cut-off value in 35% of the tested samples.

However, in the control group no patient had a positive result for 3 or 4 markers. 10% of the patients had no increase at all for any of the markers, 30% had an increase in one marker and 60% had an increase in two markers at the time of admission.

The result is again summarized in the following table:

| | Positive Marker | | | | |
|---|---|---|---|---|---|
| | 0 | 1 | 2 | 3 | 4 |
| MI group (n = 19) | 10% | 25% | 30% | 20% | 15% |
| control group (n = 20) | 10% | 30% | 60% | 0% | 0% |

A myocardial infarction can already be diagnosed on admission for 35% of the patients (all troponin negative) by determining all four parameters and using the criterium that at least 3 of the markers must be elevated.

### Example 3

Determination of a cardiospecific neurohormonal marker (NT-pro-BNP) together with a non-cardiospecific ischemic marker (IMA) for the specific diagnosis of an ischemia/unstable angina pectoris

A positive IMA value is indicative for an ischemia of unclear origin. If one considers the group of patients described above (Tables 1A and 1B) with regard to ischemia, then a diagnosis (MI or UAP) would be made for 34 patients most of which had an ischemic pathophysiology. Six patients had no ischemia as the final diagnosis (patients 23, 30, 32, 33, 36, 39=control group). However, 4 of these patients exhibited an elevated IMA value (cut-off value: 85 U/ml) (patients 23, 30, 32, 39=“false-positive”). If a positive NT-proBNP value is additionally integrated into the diagnosis (cut-off value: 350 pg/ml) the specificity increases considerably i.e. false-positive cases are no longer observed. This result is summarized in the following table.

| Positive Marker | IMA | IMA + NT-proBNP |
|---|---|---|
| MI, UAP group (n = 34) | 62% | 29% |
| control group (n = 6) | 67% | 0% |

TABLE 1A
Values for the markers of the examined group of patients (MI
Group)
MI (mycocardial infarction) group
Confirmed Cardiac Infarction
(First determination troponin T negative,
Second determination troponin T positive))

| | Pat. No/ | NT-proBNP | IMA | Myoglobin | CK-MB |
|---|---|---|---|---|---|
| Diagnosis | sample | [pg/ml] | [U/ml] | [ng/ml] | [U/l] |
| MI | 1 | 106 | 100 | 43 | 5 |
| MI | 2 | 398 | 80 | 29 | 5 |
| MI | 3 | 4663 | 107 | 207 | 23 |
| MI | 4 | 68 | 82 | 71 | −6 |
| MI | 5 | 11222 | 78 | 60 | 18 |
| MI | 6 | 105 | 120 | 43 | 5 |
| MI | 7 | 158 | 74 | 28 | 6 |
| MI | 8 | 16 | 115 | 32 | 6 |
| MI | 9 | 291 | 76 | 239 | 20 |
| MI | 10 | 15364 | 111 | 152 | 30 |
| MI | 11 | 147 | 100 | 107 | 3 |
| MI | 12 | 29 | 71 | 28 | 9 |
| MI | 13 | 189 | 110 | 955 | 32 |
| MI | 14 | 825 | 95 | 44 | 17 |
| MI | 15 | 62 | 101 | 98 | 6 |
| MI | 16 | 460 | 105 | 68 | 10 |
| MI | 17 | 1123 | 95 | 27 | 3 |
| MI | 18 | 154 | 94 | 48 | 6 |
| MI | 19 | 342 | 70 | 739 | 18 |
| MI | 20 | 79 | 80 | 35 | 3 |

TABLE 1B
Values for the markers of the examined group of patients
(Control Group)
Control group
no cardiac infarction
(first and second determination troponin T negative)

| | | NT- | | | |
|---|---|---|---|---|---|
| | Pat. No./ | proBNP | IMA | Myoglobin | CK-MB |
| Diagnosis | sample | [pg/ml] | [U/ml] | [ng/ml] | [U/l] |
| UAP = unstable | 21 | 1125 | 83 | 17 | 10 |
| angina pectoris | | | | | |
| UAP | 22 | 4022 | 93 | 20 | 6 |
| thorax pain at rest | | | | | |
| thoracic spine | 23 | 212 | 100 | 25 | 8 |
| syndrome | | | | | |
| UAP | 24 | 35 | 84 | 23 | 7 |
| UAP | 25 | 458 | 97 | 33 | 8 |
| UAP | 26 | 84 | 92 | 50 | 5 |
| UAP | 27 | 2212 | 108 | 61 | 7 |
| UAP | 28 | 55 | 91 | 39 | 5 |
| UAP | 29 | 845 | 103 | 54 | 7 |
| thorax pain at rest | 30 | 181 | 107 | 19 | 1 |
| UAP | 31 | 660 | 102 | 24 | 9 |
| thorax pain at rest | 32 | 32 | 107 | 15 | 8 |
| thorax pain at rest | 33 | 260 | 77 | 31 | 21 |
| UAP | 34 | 108 | 76 | 25 | 18 |
| UAP | 35 | 113 | 100 | 21 | 3 |
| left th. pain rez. | | | | | |
| gastr. | 36 | 98 | 65 | 20 | 7 |
| UAP | 37 | 328 | 66 | 35 | 13 |
| UAP | 38 | 192 | 103 | 48 | 6 |
| thorax pain at rest | | | | | |
| thoracic spine | 39 | 134 | 98 | 13 | 3 |
| syndrome | | | | | |
| UAP | 40 | 357 | 97 | 13 | 6 |

The above description is only to be considered illustrative of exemplary embodiments, which achieve the features and advantages of the present invention. Modification and substitutions to specific process steps, system, and setup can be made without departing from the spirit and scope of the present invention. Accordingly, the invention is not to be considered as being limited by the foregoing description, but is only limited by the scope of the appended claims.

What is claimed is:

1. A method for diagnosing acute myocardial, ischemic diseases in a patient, the method comprising determining in at least one patient sample:
(a) a cardiospecific neurohormonal marker, and
(b) at least one of
(i) a non-cardiospecific ischemic marker selected from the group consisting of ischemia-modified albumin (IMA), fatty acid, pregnancy associated plasma protein A, and sphingosine-1-phosphate, and
(ii) a non-cardiospecific muscle-specific marker,
and relating the presence or amount of markers (a) and (b) in the at least one patient sample to the risk or severity of acute myocardial, ischemic disease in the patient.

2. The method of claim 1 wherein the cardiospecific neurohormonal marker is selected from the group consisting of A-type natriuretic peptide (ANP), the N-terminal fragment of pro-ANP (NT-proANP), B-type natriuretic peptide (BNP) and the N-terminal fragment of pro-BNP (NT-pro-BNP).

3. The method of claim 2 wherein the neurohormonal marker is BNP or NT-proBNP.

4. The method of claim 1 wherein the non-cardiospecific muscle-specific marker is myoglobin or CK-MB.

5. The method of claim 1 further comprising the determination of a non-cardiospecific marker for platelet activation.

6. The method of claim 5 wherein the non-cardiospecific marker for platelet activation is CD40.

7. The method of claim 1 further comprising the determination of a cardiospecific ischemic-necrotic marker.

8. The method of claim 7 wherein the the cardiospecific ischemic-necrotic marker is troponin T or troponin 1.

9. The method of claim 8 wherein the the ischemic-necrotic marker is troponin T.

10. The method of claim 1 wherein the disease is acute myocardial infarction.

11. The method of claim 1 wherein the markers are determined in parallel.

12. The method of claim 11 wherein the determinations are carried out on a single patient sample.

13. The method of claim 1 wherein the determinations are carried out on an automated analyzer.

14. The method of claim 1 wherein the determinations are carried out as a rapid test.

15. The method of claim 14 wherein the the determinations are carried out on a test strip.

16. A reagent kit for the diagnosis of acute myocardial, ischemic diseases, the kit comprising detection reagents for determining (a) a cardiospecific neurohormonal marker, and (b) at least one of (i) a non-cardiospecific ischemic marker selected from the group consisting of ischemia-modified albumin (IMA), fatty acid, pregnancy associated plasma protein A, and sphingosine-1-phosphate, and (ii) a non-cardiospecific muscle-specific marker.

17. The reagent kit of claim 16 further comprising at least one of a detection reagent for a non-cardiospecific marker for platelet activation and a detection reagent for a cardiospecific ischemic-necrotic marker.

18. The reagent kit of claim 16 wherein the kit allows for the concurrent determinations of the markers.

19. The reagent kit according to claim 16 further comprising at least one test strip for carrying out the determinations.

20. A reagent kit for diagnosing acute myocardial, ischemic diseases, comprising at least one test strip comprising reagents for determining (a) at least one of BNP and NP-proBNP, and (b) IMA.

21. The reagent kit of claim 20 wherein the at least one test strip further comprises reagents for determining at least one of myoglobin, CK-MB, troponin T and CD40.
